(19) United States
(12) Patent Application Publication
(10) Pub. No.: US 2015/0059795 A1
(43) Pub. Date: Mar. 5, 2015

(54) HAIR CARE COMPOSITION COMPRISING VISCOELASTIC PARTICLES

(71) Applicant: The Procter & Gamble Company, Cincinnati, OH (US)

(72) Inventors: Michael Lee Vatter, Okeana, OH (US); Timothy James Felts, Hamilton, OH (US); Jorge Max Sunkel, Cincinnati, OH (US)

(21) Appl. No.: 14/478,013

(22) Filed: Sep. 5, 2014

Related U.S. Application Data
(60) Provisional application No. 61/874,045, filed on Sep. 5, 2013.

(51) Int. Cl. A61K 8/72; A61Q 5/12; A45D 7/06; A61K 8/89

(52) U.S. Cl. CPC A61K 8/72; A61K 8/89; A61Q 5/12; A45D 7/06; A61K 2800/654
USPC 132/206; 424/70.11; 424/70.12; 424/70.13; 424/70.14; 424/43; 424/70.17

(57) ABSTRACT

Hair care and styling compositions that can provide shine, frizz control and temporary styling benefits. More specifically, it relates to hair treatment compositions that comprise of viscoelastic particles in an aqueous carrier or a mixture of water with organic or silicone carrier.

### FIELD OF THE INVENTION

The present invention relates to a hair treatment or styling composition and methods of making and using the same. More specifically, it relates to a hair treatment or styling composition comprising viscoelastic particles exhibiting a particular storage modulus (G′) and loss modulus (G″) values.

### BACKGROUND OF THE INVENTION

Hair frizz can take a number of different forms. Hair fizz can occur when hair fibers which can be visually seen either, departing from the bulk or falling out of alignment of a desired hair style pattern. Frizz occurs due to environmental changes, such as humidity fluctuations and static electricity built-up, and as a result of a person's daily normal motion and activities that may cause hair fiber to shift and interlock.

Existing frizz control hair care compositions contain dimethicone fluids and other oils that coat the hair surface with a film causing hair fibers to stick together and align. Although these products may provide a shiny look, they also impart negative greasy look and oily feel on the hair. They also significantly increase the weight of each fiber and negatively impact hair volume and ease of styling. Therefore, it is desired to have a hair care composition which can deliver the benefits of frizz control without the associated negative trade-offs of greasy look and oily feel.

Hair fiber alignment can also impact the shiny appearance of hair. Hair fibers that are aligned parallel to each other show a minimum scattering of light as well as increased specular light reflection. A material that can adhere well to hair fibers, creating a bridge between neighboring fibers results in such alignment, thus improving the appearance of shiny hair. The hair care composition described herein can create relatively durable attachment to hair fibers or, alternatively, it may be removed and re-attached to different pairs or hair fibers at different times.

Additionally, hair care compositions that can distribute numerous particles of the material across consumer's hair may also have a significant positive effect on hair shine.

Traditionally, hair care compositions that deliver shine and control frizz are based on oils and silicone fluids. These types of treatments can create a negative trade-off of greasy look and oily feel. Most commercially-available compositions claiming anti-frizz benefits do not perform near a satisfactory level and consumers complain about the trade-off between long-lasting performance, feel and appearance they have to navigate through to achieve their desired look.

Including the viscoelastic particles described herein in a hair care composition can create effective alignment by bridging hair fibers without the need for fiber coating. The fact that the fibers can be aligned at larger distances with the use of such viscoelastic particles can provide shine with no detriment to hair volume. An additional benefit of this viscoelastic particle is its ability to provide a soft smooth feel to the hair. Further, the solvent and carrier in the hair care composition evaporates leading to less material staying on the hair, reducing weigh down and maintaining hair volume.

### SUMMARY OF THE INVENTION

A hair care composition comprising: from about 1 wt % to about 80 wt % of viscoelastic particles, wherein the viscoelastic particles comprise from about 0.5 wt % to about 50 wt % crosslinked polymer swelled in about 50 wt % to about 99.5 wt % volatile solvent; from about 99 wt % to about 20 wt % of a carrier; wherein the viscoelastic particle has a dried storage modulus of about 85 to about 10,000 Pascals, and a dried loss modulus of about 50 to about10,000 Pascals; and wherein the carrier comprises less than about 2 wt % non-volatile solvent.

The composition minimizes any excipient materials that may lead to weigh down and less volume.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1 is an SEM image of polymer bridges formed between hair fibers.

FIG. 2 is a model of fiber adhesion test method.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights percents (wt %) as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The term “substantially free from” or “substantially free of” as used herein means less than about 2%, or less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, and/or alcohol derivatives of a given compound.

“Polymer,” as used herein, means a chemical formed from the polymerisation of two or more monomers, which may be the same or different. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

The term “hair treatment composition” includes leave-on product compositions comprising a viscoelastic particle. The hair treatment composition may further comprise at least one other non-polymeric active ingredients or additives chosen from scalp care substances, hair care substances, photoprotective substances, oils, waxes, preservatives, pigments, soluble dyes, particulate substances, and surfactants in a suitable cosmetic base. The hair-treatment composition can be in the form typically used as a leave-on hair treatment such as a gel, a lotion, a spray, a cream, or a foam.

The features of the composition according to the first aspect, as well as the other aspects and other relevant components, are described in detail hereinafter. All components of the composition described herein should be physically and chemically compatible with the essential components described herein, and should not otherwise unduly impair composition stability, aesthetics or performance.

### Hair Care Composition

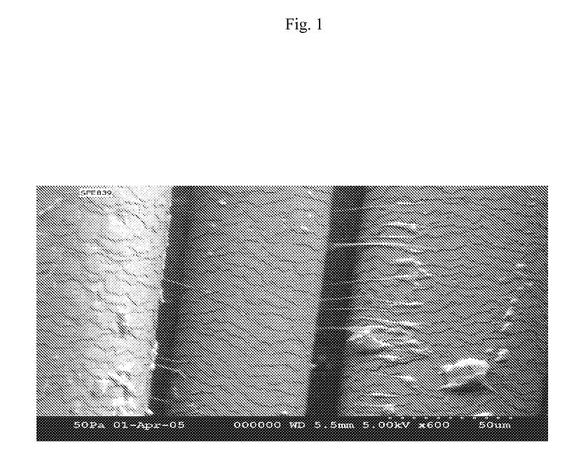

Typical dimethicone fluids coat the hair surface in a thin film causing hair fibers to stick together in a tight fashion. This can create weigh down of the hair and result in less volume and a greasy look. The hair care compositions described herein comprise a viscoelastic particle which forms bridges between fibers increasing the fiber to fiber separation and leading to increased volume. See FIG. 1.

The viscoelastic particles of the present invention typically consist of a crosslinked polymer or combination of crosslinked polymers and a swelling solvent system. The solvent system is compatible with the crosslinked polymer, it penetrates the polymer particle and swells it, but it does not dissolve it or it does not dissolve in the polymer to solvent ratio used. If part, or all, of the solvent(s) used are volatile, a portion or all of the solvent may evaporate over time, resulting in dried polymer with different viscoelastic properties. This allows one to design a hair care composition that provides improved benefits because of the optimized properties of the viscoelastic particles before and after partial or complete solvent evaporation. Dried polymer measurements can be obtained by placing a 7 mm layer of the viscoelastic particles (as supplied by manufacturer) in an shallow dish and allowed to dry (or partially dry) for 5 days at ambient temperature.

The hair care composition comprises 1) from about 1% to about 80 wt % of a viscoelastic particle (from about 0.5 to about 50% crosslinked polymer and from about 50% to about 99.5% volatile solvent;) 2) from about 99% to about 20 wt % a carrier (from about 0 to about 2% non-volatile solvent); wherein the polymer is a removable Pressure Sensitive Adhesive (“PSA”) and the dried storage modulus (G′) of the crosslinked polymer is from about 85 to about 10,000 Pascals, and the dried loss modulus (G″) of the crosslinked polymer is from about 50 to about 10,000 Pascals. Materials that fall outside this description are either too fluid, too high viscosity or too elastic as shown in Comparative Examples B and C, formulas which falls outside the removable PSA and into a non-PSA region do not provide the necessary adhesive properties to result in the optimum look (alignment and not greasy) and feel (soft smooth) benefits. Additionally, it has been found that viscoelastic particles that are swelled in non-volatile solvents create a greasy appearance on hair at the levels claimed (Comparative Example D). Further, viscoelastic particles having too high or too low a dried G′ do not provide the optimum elasticity to adhere the fibers together. Therefore, failing to create the alignment and volume benefits desired by the consumer. As shown in Example B, viscoelastic particle DC9040 available from Momentive, INCI name dimethicone/vinyl dimethicone crosspolymer has a dried G′ of 10,922-15,250 Pascals and a dried G″ of 804-1907 Pascals does not form a bridge between keratin fibers. Commercially available leave on conditioning treatments containing silicone fluids such as dimethicone, dimethiconol, amino silicones, functionalized silicones, do not contain viscoelastic particles described herein and therefore they do not deliver both frizz and shine control without resulting in hair weigh down and greasy feel and look. As shown in the results included in Table 2.

A. Viscoelastic Particle

The viscoelastic particle containing a crosslinked polymer and a swelling solvent can be added to a hair care compositions, such as hair treatment or hair styling composition, at a level of crosslinked polymer from about 0.01% to about 5%, and/or from about 0.1% to about 2% by weight of the hair treatment composition. Low levels of the crosslinked polumer provides reduced hair benefit, whereas high concentrations can result in hair weigh-down as well as aesthetic negatives such as residue on hair.

The viscoelastic particle includes crosslinked organopolysiloxane or another crosslinked polymer or elastomer. Examples of suitable polymers include, but are not limited to, addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane; condensation-curing organopolysiloxane compositions which cure in the presence of an organotin compound or a titanate ester, by a condensation reaction between an hydroxyl-terminated diorganopolysiloxane and a hydrolyzable organosilane (this condensation reaction is exemplified by dehydration, alcohol-liberating, oxime-liberating, amine-liberating, amide-liberating, carboxyl-liberating, and ketone-liberating reactions); peroxide-curing organopolysiloxane compositions which thermally cure in the presence of an organoperoxide catalyst; and organopolysiloxane compositions which are cured by high-energy radiation, such as by gamma-rays, ultraviolet radiation, or electron beams, and/or combinations thereof

Addition reaction-curing organopolysiloxane compositions suitable due to their rapid curing rates and excellent uniformity of curing. A suitable addition reaction-curing organopolysiloxane composition is prepared from: (A) an organopolysiloxane having at least 2 lower alkenyl groups in each molecule; (B) an organopolysiloxane having at least 2 silicon-bonded hydrogen atoms in each molecule; and(C) a platinum-type catalyst.

With regard to the above, component (A) is the basic component of the silicone polymer-generating organopolysiloxane, and curing proceeds by the addition reaction of this component with component (B) under catalysis by component (C). Component (A) contains at least 2 silicon-bonded lower alkenyl groups in each molecule; an excellent cured product will not be obtained at fewer than two lower alkenyl groups because a network structure will not be formed.

Said lower alkenyl groups are exemplified by vinyl, allyl, and propenyl. While the lower alkenyl groups can be present at any position in the molecule, in one embodiment they are present at the molecular terminals. The molecular structure of this component may be straight chain, branched straight chain, cyclic, or network,however, in one embodiment the molecular structure is a straight chain, possibly slightly branched. The molecular weight of the component is not specifically restricted, and thus the viscosity may range from low viscosity liquids to very high viscosity gums. In order for the cured product to be obtained in the form of the rubbery elastomer, the viscosity at 25 (degree) C. can be at least about 100 centistokes. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3,-trifluoropropyl)siloxane copolymers.

Component (B) is an organopolysiloxane having at least 2 silicon-bonded hydrogen atoms in each molecule and is a crosslinker for component (A). Curing proceeds by the addition reaction of the silicon-bonded hydrogen atoms in this component with the lower alkenyl groups in component (A) under catalysis by component (C). This component (B) contains at least 2 silicon-bonded hydrogen atoms in each molecule in order to function as a crosslinker. Furthermore, the sum of the number of alkenyl groups in each molecule of component (A) and the number of silicon-bonded hydrogen atoms in each molecule of component (B) is to be at least about 5. Values below 5 should be avoided because a network structure is then essentially not formed.

No specific restriction exists on the molecular structure of this component, and it may be any of straight chain, branch-containing straight chain, cyclic, etc. The molecular weight of this component is not specifically restricted, but in one embodiment the viscosity at 25 (degree) C. be about 1 to about 50,000 centistokes in order to obtain good miscibility with component (A). In one embodiment this component is added in a quantity such that the molar ratio between the total quantity of silicon-bonded hydrogen atoms in the instant component and the total quantity of all lower alkenyl groups in component (A) falls within the range of about 1.5:1 to about 20:1. It is difficult to obtain good curing properties when this molar ratio falls below about 0.5:1. When about 20:1 is exceeded, there is a tendency for the hardness to increase to high levels when the cured product is heated. Furthermore, when an organosiloxane containing substantial alkenyl is supplementarily added for the purpose of; for example, reinforcement, a supplemental addition of the instant SiH-containing component can be made in a quantity offsetting these alkenyl groups. Suitable examples include, but are not limited to, trimethylsiloxy-terminated methylhydrogenpolysiloxanes, trimethylsiloxy-terminated dimethylsiloxane-methylhydrogensiloxane copolymers, and dimethylsiloxane-methylhydrogen-siloxane cyclic copolymers.

Component (C) is a catalyst of the addition reaction of silicon-bonded hydrogen atoms and alkenyl groups, and suitable examples include, but are not limited to, chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

This component can be added at about 0.1 to about 1,000 weight parts, and at about 1 to about 100 weight parts, as platinum-type metal proper per 1,000,000 weight parts of the total quantity of components (A) plus (B). Other organic groups which may be bonded to silicon in the organopolysiloxane forming the basis for the above-described curable organopolysiloxane compositions are, for example, alkyl groups such as methyl, ethyl, propyl, butyl, and octyl; substituted alkyl groups such as 2-phenylethyl, 2-phenylpropyl, and 3,3,3-trifluoropropyl; aryl groups such as phenyl, tolyl, and xylyl; substituted aryl groups such as phenylethyl; and monovalent hydrocarbon groups substituted by, for example, the epoxy group, the carboxylate ester group, the mercapto group, etc.

Examples of the production of the organopolysiloxane powder include, but are not limited to: an organopolysiloxane composition as described above (additional-curable, condensation-curable, or peroxide-curable) is mixed with water in the presence of a surfactant (nonionic, anionic, cationic, or amphoteric), and, after mixing to homogeneity in a homomixer, colloid mill, homogenizer, propeller mixer, etc., this is cured by discharge into hot water (temperature at least 50 (degree) C.) and is then dried; the organopolysiloxane composition (addition-curable, condensation-curable, or peroxide-curable) is cured by spraying it directly into a heated current; the powder is obtained by curing a radiation-curable organopolysiloxane composition by spraying it under high energy radiation; the organopolysiloxane composition (addition-curable, condensation-curable, peroxide-curable) or high energy-curable organopolysiloxane composition is cured, the latter by high energy radiation, and the product is then pulverized using a known pulverizer such as, for example, a ball mill, atomizer, kneader, roll mill, etc., to thereby form the powder. Suitable organopolysiloxane elastomer powders include, but are not limited to, vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200, and hybrid silicone powders that contain a phenyl group such as Shin-Etsu's KSP-300; and Dow Corning's DC 9506.

Suitable organopolysiloxane compositions include, but are not limited to, dimethicone/vinyl dimethicone crosspolymers. Such dimethicone/vinyl dimethicone crosspolymers are supplied by a variety of suppliers including Momentive (SFE 839), Shin Etsu (KSG-15, 16, 18 dimethicone/phenyl vinyl dimethicone crosspolymer), Grant Industries (Gransil Polysiloxane-11 (tm) line of materials), and lauryl dimethicone/vinyl dimethicone crosspolymers supplied by Shin Etsu (e.g., KSG-31, KSG-32, KSG-41, KSG-42, KSG-43, and KSG-44). Cross-linked organopolysiloxane elastomers useful in the present invention and processes for making them are further described in U.S. Pat. No. 4,970,252 to Sakuta et al., issued Nov. 3, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo K K, each of which are herein incorporated by reference in its entirety. Silicone elastomers of the type described in U.S. Pat. No. 5,412,004 (issued May 2, 1995); U.S. Pat. No. 5,837,793 (issued Jan. 17, 1998); and U.S. Pat. No. 5,811,487 (issued Sep. 22, 1998), all of which patents are herein incorporated by reference in their entirety, are also useful herein. In one embodiment the elastomers of the described herein are cured under anhydrous conditions or in an anhydrous environment.

The organopolysiloxane elastomers of the present invention can be further processed by subjecting them to a high shear (approximately 5,000 psi) treatment in the presence of a solvent for the siloxane elastomer via a Sonolator at less than 10 passes. Sonolation achieves a resultant composition with elastomer average particle size ranging from above 10 (or above about 10) microns to about 200 microns, from about 20 to about 150 microns, from above 30 (or above about 30) to about 100 microns, from about 40 microns to about 95 microns, and/or, from above 50 microns to about 90 microns as measured by the Horiba LA-910 (described below). As used herein, the term “particle size” of the elastomer represents the elastomer particle size in its swelled state. By “swelled,” as used herein, means that the elastomer particles have extended beyond their normal size and shape by virtue of their absorption of the solvent compound. Viscosity is best when ranging from above about 20,000 and about 6,000,000, from about 30,000 to about 4,000,000, from about 40,000 to about 3,000,000, from about 60,000 to about 2,000,000, and/or from about 70,000 to about 1,500,000 cps at 25 (degree) C. as measured by a Brookfield LV Viscometer (size 4 bar, 60 rpm, 0.3 sec.).

Without being limited by theory, it is believed that compositions incorporating elastomer/solvent gels where the elastomer has an average particle size greater than 10 microns (or greater than about 10 microns) and/or viscosities greater than about 20,000 cps provide films having improved smoothness as well as improved uniformity and evenness of particle (e.g., pigments) distribution within the film (i.e., solid particles remain distributed within and throughout the film as opposed to such particles protruding from the film into and/or across the film/air interface).

In one embodiment the organopolysiloxane elastomers do not undergo recycled processing. Without being limited by theory, recycled processing produces broad particle size distributions comprising particles larger or smaller than that necessary to achieve the skin feel benefits of the present invention. Specifically, gel balls often result from silicone elastomer particles larger than about 200 microns while elastomer particles smaller than about 10 microns reduce skin feel and viscosity benefits. Such particle size distributions result from a failure to ensure that all of the elastomer particle materials experience the same shear throughout the process. Typically, with recycling, only a portion of the particles experience shear before these sheared particles are returned to the process starting point and combined with the remaining un-sheared particles. Similarly, the next cycle begins with only a portion of this particle mixture experiencing shear before the newly sheared mixture particles are returned to the process starting point and combined with the remaining un-sheared particle mixture. Even after considerable recycling, some of the particles never actually experience shear while others experience a high degree of shear. The result is a particle size range that may encompasses particles both larger and smaller than those desired to achieve the present invention.

In contrast, discrete pass processing, ensures that all the particles experience shear as well as the same amount of shear with each run or pass. More specifically, no run or pass is completed until all the particles have experienced the same shear force. Consequently, the particle size distribution is narrower than that produced by “recycling” with respect to specific particle sizes. This results in a better balance between gel ball formation and viscosity as well as skin feel and viscosity.

In one embodiment the viscoelastic particle is non-emulsifying. The term “non-emulsifying,” as used herein, defines organopolysiloxane elastomer from which polyoxyalkylene units are absent.

In one embodiment the viscoelastic particle is non-spherical. By the term “non-spherical” as used herein means that the siloxane elastomer particles are not spherical, and not spheroidal. Without being limited by theory, the present inventors believe that spherical particles fail to provide the rheology and film properties desired to achieve the benefits of the present invention. Specifically, when forming the gel matrix or network, spherical particles do not swell to the extent and/or pack as tightly as non-spherical particles.

Amounts of the elastomer may range from about 0.1 to about 10%, from about 1 to about 8%, and/or from about 3 to about 6% by weight.

B. Solvent for the Viscoelastic Particle

The compositions of the present invention comprise a solvent for the viscoelastic particle. The solvent, when combined with the crosslinked polymer, serves to suspend and swell the polymer particles to provide an elastic, gel-like network or matrix. The solvent for the viscoelastic particle is liquid under ambient conditions, and preferably has a low viscosity to provide for improved spreading on the skin.

Concentrations of the solvent in the hair care compositions of the present invention will vary primarily with the type and amount of solvent and the crosslinked polymer employed. Suitable concentrations of the solvent are from about 10% to about 95%, from about 20% to about 80%, from about 30% to about 70%, by weight of the composition.

The solvent for the viscoelastic particle comprises one or more liquids suitable for topical application to human hair and skin. The solvent can include volatile, non-polar oils; non-volatile, relatively polar oils; non-volatile, non-polar oils; and non-volatile paraffinic hydrocarbon oils. The term “non-volatile” as used herein refers to materials which exhibit a boiling point at one atmosphere of at least about 250 (degree) C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm3) 0.5. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988, which articles are incorporated herein by reference.

### 1. Non-Polar, Volatile Oils

The non-polar, volatile oil tends to impart desirable aesthetic properties to the hair care composition described herein. Consequently, the non-polar, volatile oils are utilized at a fairly high level. Non-polar, volatile oils particularly useful in the present invention are selected from the group consisting of silicone oils; hydrocarbons; and mixtures thereof. Such non-polar, volatile oils are disclosed, for example, in Cosmetics, Science, and Technology, Vol. 1, 27-104 edited by Balsam and Sagarin, 1972. Suitable non-polar, volatile oils include those that are either saturated or unsaturated, have an aliphatic character and straight or branched chained or contain alicyclic or aromatic rings. Examples of suitable non-polar, volatile hydrocarbons include, but are not limited to, polydecanes such as isododecane and isodecane (e.g., Permethyl-99A which is available from Presperse Inc.) and the C7-C8 through C12-C15 isoparaffins (such as the Isopar Series available from Exxon Chemicals). Non-polar, volatile liquid silicone oils are disclosed in U.S. Pat. No. 4,781,917 issued to Luebbe et al. on Nov. 1, 1988, herein incorporated by reference in its entirety. Additionally, a description of various volatile silicones materials is found in Todd et al., “Volatile Silicone Fluids for Cosmetics”, Cosmetics and Toiletries, 91:27-32 (1976), herein incorporated by reference in its entirety. In one embodiment the volatile silicone oils are selected from the group consisting of cyclic volatile silicones and linear volatile silicones. Linear volatile silicones generally have a viscosity of less than about 5 centistokes at 25 (degree) C., whereas the cyclic silicones have viscosities of less than about 10 centistokes at 25 (degree) C. Suitable examples of volatile silicone oils include cyclomethicones of varying viscosities, e.g., Dow Corning 200, Dow Corning 244, Dow Corning 245, Dow Corning 344, and Dow Corning 345, (commercially available from Dow Corning Corp.); SF-1204 and SF-1202 Silicone Fluids (commercially available from G.E. Silicones), GE 7207 and 7158 (commercially available from General Electric Co.); and SWS-03314 (commercially available from SWS Silicones Corp.).

### 2. Relatively Polar, Non-Volatile Oils

The non-volatile oil is “relatively polar” as compared to the non-polar, volatile oil discussed above. Therefore, the non-volatile co-solvent is more polar (i.e., has a higher solubility parameter) than at least one of the non-polar, volatile oils. Relatively polar, non-volatile oils potentially useful in the present invention are disclosed, for example, in Cosmetics, Science, and Technology, Vol. 1, 27-104 edited by Balsam and Sagarin, 1972; U.S. Pat. Nos. 4,202,879 issued to Shelton on May 13, 1980; and 4,816,261 issued to Luebbe et al. on Mar. 28, 1989, all of which are herein incorporated by reference in their entirety. Relatively polar, non-volatile oils useful in the present invention are selected from the group consisting of silicone oils; hydrocarbon oils; fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof. The relatively polar, non-volatile co-solvents useful in the present invention may be either saturated or unsaturated, have an aliphatic character and be straight or branched chained or contain alicyclic or aromatic rings. In one embodiment, the relatively polar, non-volatile liquid co-solvent are selected from the group consisting of fatty alcohols having from about 12-26 carbon atoms; fatty acids having from about 12-26 carbon atoms; esters of monobasic carboxylic acids and alcohols having from about 14-30 carbon atoms; esters of dibasic carboxylic acids and alcohols having from about 10-30 carbon atoms; esters of polyhydric alcohols and carboxylic acids having from about 5-26 carbon atoms; ethoxylated, propoxylated, and mixtures of ethoxylated and propoxylated ethers of fatty alcohols with from about 12-26 carbon atoms and a degree of ethoxylation and propoxylation of below about 50; and mixtures thereof. Additional suitable solvents include propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv TN supplied by Finetex), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof. Additional suitable solvent include branched-chain aliphatic fatty alcohols having from about 12-26 carbon atoms. Examples include, but are not limited to, isocetyl alcohol, octyidecanol, octyldodecanol and undecylpentadecanol; and in one embodiment the solvent includes octyldodecanol. Such aliphatic fatty alcohols are particularly useful in combination with the volatile liquid silicone oils discussed herein to adjust the average solubility of the solvent.

### 3. Non-Polar, Non-Volatile Oils

In addition to the liquids discussed above, the solvent for the cross-linked siloxane elastomer may optionally include non-volatile, non-polar oils. Typical non-volatile, non-polar emollients are disclosed, for example, in Cosmetics, Science, and Technology, Vol. 1, 27-104 edited by Balsam and Sagarin, 1972; U.S. Pat. No. 4,202,879 issued to Shelton on May 13, 1980; and U.S. Pat. No. 4,816,261 issued to Luebbe et al. on Mar. 28, 1989, both of which are herein incorporated by reference. The non-volatile oils useful in the hair care composition described herein are essentially non-volatile polysiloxanes, paraffinic hydrocarbon oils, and mixtures thereof. The polysiloxanes useful in the present invention are selected from the group consisting of polyalkylsiloxanes, polyarylsiloxanes, polyalkylarylsiloxanes, poly-ethersiloxane copolymers, and mixtures thereof. Examples of these include polydimethyl siloxanes having viscosities of from about 1 to about 100,000 centistokes at 25 (degree) C. Among the non-volatile silicone emollients useful in the present compositions are the polydimethyl siloxanes having viscosities from about 2 to about 400 centistokes at 25 (degree) C. Such polyalkylsiloxanes include the Viscasil series (sold by General Electric Company) and the Dow Corning 200 series (sold by Dow Corning Corp.). Polyalkylarylsiloxanes include polymethylphenyl siloxanes having viscosities of from about 15 to about 65 centistokes at 25 (degree) C. These are available, for example, as SF 1075 methyl-phenyl fluid (sold by General Electric Company) and 556 Cosmetic Grade Fluid (sold by Dow Corning Corp.). Useful polyethersiloxane copolymers include, for example, a polyoxyalkylene ether copolymer having a viscosity of about 1200 to 1500 centistokes at 25 (degree) C. Such a fluid is available as SF1066 organosilicone surfactant (sold by General Electric Company). Polysiloxane ethylene glycol ether copolymers are suitable copolymers for use in the present compositions.

Non-volatile paraffinic hydrocarbon oils useful in the present invention include mineral oils and certain branched-chain hydrocarbons. Examples of these fluids are disclosed in U.S. Pat. No. 5,019,375 issued to Tanner et al. on May 28, 1991, herein incorporated by reference in its entirety. Suitable mineral oils have the following properties: (1) viscosity from about 5 centistokes to about 70 centistokes at 40 (degree) C.; (2) density between about 0.82 and 0.89 g/cm3 at 25 (degree) C.; (3) flash point between about 138 (degree) C. and about 216 (degree) C.; and (4) carbon chain length between about 14 and about 40 carbon atoms.

Suitable branched chain hydrocarbon oils have the following properties: (1) density between about 0.79 and about 0.89 g/cm3 at 20 (degree) C. (2) boiling point greater than about 250 (degree) C.; and (3) flash point between about 110 (degree) C. and about 200 (degree) C.

Additional suitable branched-chain hydrocarbons include Permethyl 103A, which contains an average of about 24 carbon atoms; Permethyl 104A, which contains an average of about 68 carbon atoms; Permethyl 102A, which contains an average of about 20 carbon atoms; all of which may be purchased from Permethyl Corporation; and Ethylflo 364 which contains a mixture of 30 carbon atoms and 40 carbon atoms and may be purchased from Ethyl Corp.

When used herein, volatile or non-volatile hydrocarbon oils are present at concentrations less than 50%, from about 1% to about 30%, and/or from about 0% to about 20%.

Additional solvents useful herein are described in U.S. Pat. No. 5,750,096 to Gerald J. Guskey et al., issued May 12, 1998, herein incorporated by reference in its entirety.

Other (non-silicone-based) elastomeric or other polymeric materials can be used in another embodiment of this inventions such as: Polyolefin gels, polyacrylate copolymers and emulsions, styrene/acrylate copolymers, and polyurethane-14/AMP acrylate copolymers. Non-limited examples include polyolefin oil gel, Epitex-66, supplied by Dow Chemical, and DynamX supplied by Akzo Nobel. Other suitable dimethicone polymers include GI CD-10, GI CD-11, Gransil DMG-2, Gransil DMG-3, Gransil DMG-5, Gransil GCM-5, Gransil GTS, Gransil GVL, Gransil GVL-HV, Gransil IDS-5, Gransil MLB, Gransil PC-12, Gransil PC-12P, Gransil RPS, Gransil RPS-D6, Gransil DM-5, Gransil DMCM-5, Gransil DMDM-25, Gransil DMDM-35, Gransil DMID, Gransil DMTS, Gransil GAM, Gransil MLB-SCS, Gransil DM-10, Gransil DMAM, Gransil DMG-20, Gransil DMG-6, Gransil PM, Gransil OHS-5, Gransil PS-5, Granactive AA-20, Granactive Hexapeptide, Gransil DM-5/AA, Gransil OHS-5/AA, Gransil PS-5/AA-20 from Grant industries, Y17204, Silsoft 1215, Silsoft ETS, Silformflex, from Momentive, Dc9040, DC9546, EL-7040, 2-2078, PH-1560, DC-3055, Accudyne Shine from Dow Corning, Glossyliance from Soliance, KSG-820Z, KSG-210, KSG-43, KSG-340, KSG-330, X22-6611A, from Shin-Etsu, and AbilCare 85 from Evonic.

C. Thickener

In one embodiment the hair care composition includes rheology modifiers to adjust the rheological characteristics of the composition for better feel, in-use properties and and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. In an embodiment, the hair care composition may comprise from about 0.01% to about 3% of a rheology modifier, alternatively from about 0.1% to about 1% of a rheology modifier,

The rheology modifier may be a polyacrylamide thickener, a cationically modified polysaccharide, or an associative thickeners. Associative thickeners is an important class of rheology modifiers. It includes a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. The class of materials includes numerous members. Typically these materials have a hydrophobe that can be selected from cetyl, stearyl, oleayl, and combinations thereof, and a hydrophilic portion of repeating ethylene oxide groups with repeat units from 10-300, more preferably from 30-200, more preferably from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of rheology modifiers include acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

D. Carrier

Hair care compositions typically comprise a carrier, which is present at a level of from about 20 wt % to about 99 wt %, and/or from about 60 wt % to about 85 wt %. The carrier may comprise water, organic solvents (miscible or non-miscible with water), silicone solvents and/or a mixtures thereof. The solvents should be dermatologically acceptable. The carrier does not comprise more than about 2, about 1, about 0.5, about 0.2, about 0.1, and/or about 0.05 wt % of non-volatile solvent. Significantly higher concentration of non-volatile carrier will increase hair weigh-down. and greasy hair feel. In one embodiment the carrier may comprise water with minimal or no significant concentrations of organic solvent, except as otherwise incidentally incorporated into the composition as minor ingredients of other components. Water, organic and silicone solvents that have boiling points below or equal to 250° C. are volatile solvents. Solvents with boiling points above 250° C. are considered non-volatile.

The carrier useful in embodiments of the hair care composition includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include glycols, glycerine and other diols.

E. Optional Ingredients

The hair treatment and/or styling compositions of the present invention can also additionally comprise any suitable optional ingredients as desired. Such optional ingredients should be physically and chemically compatible with the components of the composition, and should not otherwise unduly impair composition stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

The composition of the present invention can include a styling polymer. A styling polymer may be selected from the group consisting of acrylate polymers and their esters, methacrylate polymers and their esters, acrylate copolymers and their esters, methacrylate copolymers and their esters, polyureathane polymers and copolymers, polyvinylpyrrolidones (PVP), PVP-polyvinyl acetate copolymers, PVP-polivinyl alcohol copolymers, polyesters, and other polymers.

Additional optional ingredients include, but are not limited to: anti-dandruff actives, antimicrobial materials, skin and scalp actives, vitamins, hair growth actives, conditioning materials, fragrances, pigments, colorings/colorants, essential oils, sensates, etc., antifoaming agents, antioxidants, binders, biological additives, buffering agents, bulking agents, chelating agents, chemical additives, film formers or materials, opacifying agents, pH adjusters, propellants, reducing agents, and surfactants.

F. Composition Form

In one embodiment, the hair care composition is in the form of an O/W emulsion, a W/O emulsion or a microemulsion.

In one embodiment, the hair care composition is in the form of a spray composition, either in combination with a mechanical pump spray device or in combination with at least one propellant chosen from propane, butane, dimethyl ether and fluorinated hydrocarbons. An aerosol spray can additionally comprise about 15 to about 85% by weight, particularly about 25 to about 75% by weight, of a propellant and is bottled in a pressurized container. Suitable propellants are, for example, lower alkanes, such as, for example, n-butane, isobutane and propane, or mixtures thereof, and dimethyl ether or fluorinated hydrocarbons, such as F 152a (1,1-difluoroethane) or F 134 (tetrafluoroethane), and also propellants which are in gaseous form at the pressures under consideration, such as, for example, N2, N20 and CO2, and mixtures of the abovementioned propellants.

A nonaerosol hairspray is sprayed with the help of a suitable mechanically operated spraying device. Mechanical spraying devices are understood as meaning those devices which permit the spraying of a composition without use of a propellant. A suitable mechanical spray device used may, for example, be a spray pump or an elastic container provided with a spray valve in which the cosmetic agent according to the invention is bottled under pressure, where the elastic container expands and from which the agent is continuously dispensed as a result of the elastic container contracting upon opening the spray valve.

In one embodiment, the hair care composition is in the form of a foamable composition (mousse) in combination with a devices for foaming, comprises at least one customary foam-imparting substance known for this purpose, e.g. at least one foam-forming surfactant or at least one foam-forming polymer. Devices for foaming are understood as meaning those devices which enable the foaming of a liquid with or without use of a propellant. A suitable mechanical foaming device which can be used is, for example, a standard commercial pump foamer or an aerosol foaming head. The composition is either in combination with a mechanical pump foaming device (pump foam) or in combination with at least one propellant (aerosol foam) in an amount of from about 1 to about 20% by weight, in another embodiment from about 2 to about 10% by weight. Propellants are chosen, for example, from propane, butane, dimethyl ether and fluorinated hydrocarbons. The hair care composition is foamed directly prior to application and incorporated into the hair as foam and can then be rinsed out or left in the hair without rinsing.

The foamable compositions comprise, as active ingredients or additives, in one embodiment, polymers which are chosen from chitosan, chitosan salts, chitosan derivatives, cationic cellulose compounds, copolymers of vinylpyrrolidone, vinylcaprolactam and a basic acrylamide monomer or mixtures of these polymers. Suitable chitosan salts, chitosan derivatives, cationic cellulose derivatives are, for example, those mentioned above. Suitable cationic cellulose compounds include, but are not limited to, copolymers of hydroxyethylcellulose and diallyldimethylammonium chloride (polyquaternium-4) and reaction products of hydroxyethylcellulose and epoxides substituted by a trialkylammonium group (Polyquaternium-10). Suitable chitosan salts include, but are not limited to, the salts with formic acid, lactic acid and pyrrolidonecarboxylic acid. Suitable copolymers of vinylpyrrolidone, vinylcaprolactam and a basic acrylamide monomer are those in which the acrylamide monomer is dimethylaminopropylacrylamide. One embodiment of foamable compositions comprises copolymers of hydroxyethylcellulose and diallyldimethylammonium chloride (polyquaternium-4) and copolymers of vinylpyrrolidone, vinylcaprolactam and dimethylaminopropylacrylamide, and foamable compositions which comprise copolymers of hydroxyethylcellulose and diallyldimethylammonium chloride (polyquaternium-4) and copolymers of vinylpyrrolidone, vinylcaprolactam and dimethylaminopropylacrylamide, and at least one chitosan salt.

In one embodiment, the hair care composition is in the form of a hair wax, i.e. it has a wax-like consistency and comprises at least one of the above-mentioned waxes in an amount of from about 0.5 to about 30% by weight, and optionally further water-insoluble substances. In one embodiment the wax-like consistency has a needle penetration number (unit of measurement 0.1 mm, test weight 100 g, test time 5 s, test temperature 25° C.; according to DIN 51 579) of greater than or equal to about 10, and in another embodiment greater than or equal to about 20 and that the solidification point of the composition is greater than or equal to about 30° C. and less than or equal to about 70° C., and in one embodiment in the range from about 40 to about 55° C. Suitable waxes and water-insoluble substances are, in particular, emulsifiers with an HLB value below 7, silicone oils, silicone waxes, waxes (e.g. wax alcohols, wax acids, wax esters, and in particular natural waxes such as beeswax, carnauba wax, etc.), fatty alcohols, fatty acids, fatty acid esters or hydrophilic waxes, such as, for example, high molecular weight polyethylene glycols with a molecular weight of from about 800 to about 20 000, and in one embodiment from about 2000 to about 10 000 g/mol.

If the hair care composition is in the form of a hair lotion, then it is in the form of an essentially nonviscous or low-viscosity, flowable solution, dispersion or emulsion with a content of at least about 10% by weight, in another embodiment from about 20 to about 95% by weight, of a cosmetically compatible alcohol. Alcohols which can be used are, in particular, the lower alcohols having 1 to 4 carbon atoms customarily used for cosmetic purposes, e.g. ethanol and isopropanol.

If the hair care composition is in the form of a hair cream, then it can be in the form of an emulsion and comprises either additionally viscosity-imparting ingredients in an amount of from about 0.1 to about 10% by weight, or the required viscosity and creamy consistency is built up in a customary manner through micelle formation with the help of suitable emulsifiers, fatty acids, fatty alcohols, waxes etc.

### Method of Use

The present invention also provides a method for minimizing frizz and/or increasing hair shine, without resulting in hair weigh down and/or greasy feel and look. In one aspect, the method comprises applying a hair care composition comprising a viscoelastic particle to a hair surface.

In still another embodiment, wherein said regimen comprises:
(a) applying the hair care composition comprising the viscoelastic particles to the hair,
(b) optionally styling the hair with a heat device including, but not limited to, a hair dryer, curling iron, and/or flat iron.

### Test Methods

A. Visual Evaluations

1. Switch Preparation

Switches of mildly oxidatively damaged hair (8 inches in length, 4 grams) are wetted with 100° F. water and squeegeed with the fingers to remove excess water. Apply 0.4 ml of a non-conditioning shampoo (0.2 ml per side) down length of switch and work product through switch creating lather for 30 seconds. Rinse out the shampoo with running 100° F. water. Apply desired amount (consistent quantities as described below in Table 3) of test product (half per side) down length of switch and work product through switch for 30 seconds. Leave hair to dry. Apply test product at desired amount and let dry. Comb switch and evaluate for look and feel attributes.

2. Switch Evaluation:

The panelist grade hair scores on 4 gram, 8-inch long hair switches via visual inspection, rating the hair switches on alignment, greasiness and frizz. The scale is a 1-5 scale with 1=Best and 5=Worst for alignment, greasiness, and frizz. As shown in Table 1.

TABLE 1

| Alignment | Greasiness | Frizz |
|---|---|---|
| 1 = Excellent | 1 = None | 1 = No Frizz |
| 2 = Very Good | 2 = Slight | 2 = Slight |
| 3 = Good | 3 = Minimal | 3 = Minimal |
| 4 = Fair | 4 = Moderate | 4 = Moderate |
| 5 = Poor | 5 = Heavy | 5 = Lots of Frizz |

3. Fiber Adhesion Method:

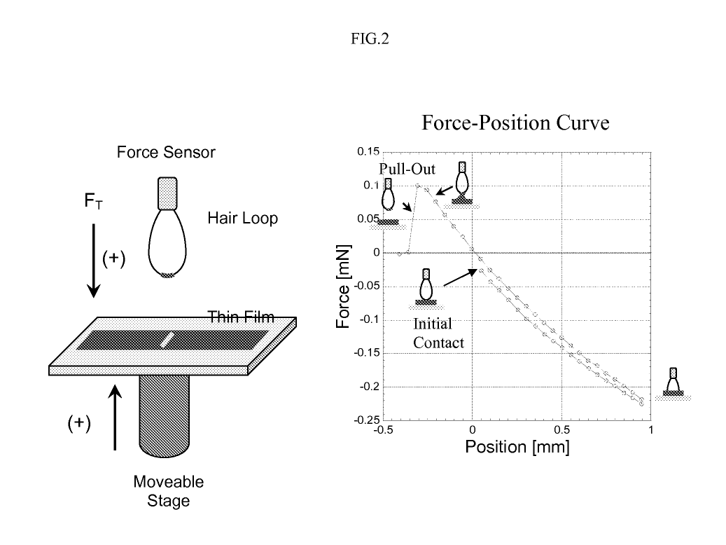

This method determines the adhesive properties between the viscoelastic particle and a hair fiber. The higher the force, the greater the adhesive properties between the hair fiber and the viscoelastic particle. A Kruss 100 tensiometer instrument is used to measure force interaction of hair against a surface, a film of a material casted on a surface or another single hair fiber. The intent for this technique is to be able to bring two surfaces in contact with each other and have very precise control as to the location of these, before and during contact, while being able to monitor the force interaction between them with a high level of precision. In order to bring these two surfaces in contact, such as a single hair fiber and a thin film of material casted on a glass slide, the motorized stage of the Kruss tensiometer is moved in the vertical direction at a specified speed, direction and location. The sensitive balance mounted on the top of the instrument is capable of reading up to 0.01 mg, and is used to determine the force interaction between the two hairs as the figure below indicates.
Equipment: Kruss 100 tensiometer (or equivalent)
a) Software is set to use the ‘Contact Angle’ module: Single fiber method. The diameter of the fiber is not used in the measurement so can be set to an arbitrary value (ex. 0.1 mm)
b) A single hair fiber loop is attached to the measurement sensor with an alligator style clip such that the ‘loose’ ends of the fiber are clamped in the clip and the ‘loop’ hangs down from the clip. The force on the sensor is set to zero.
c) A thin film of the viscoelastic particles is prepared on a glass slide by placing two pieces of adhesive tape along both long sides of a glass slide, about 1 cm apart from each other. A small amount of material is placed on the exposed glass surface in between the two tape strips, at one side of the slide. With a sharp edge object, such as a utility blade, the material is spread level with the tape height by dragging the blade across the tape forcing the material into the ‘valley’ created by the tape. This spreads the material on the exposed glass surface in between the strips, and upon the removal of the tape a film of material with uniform thickness is obtained.
d) Use double sided tape to affix the glass slide with the viscoelastic particles to the stage directly under the hanging hair fiber loop.
e) The test parameters are set such that the stage is raised to bring the hair fiber and material into contact at a rate of 1 mm/min (measurement is started when −0.025 mN force is detected on the sensor and this position is recorded as the zero position). Once measurement is started the stage is raised 1 mm at a rate range of 0.01-10 mm/sec. It is held at this position for 0-60 seconds and then lowered 1.5 mm at a rate of 0.01-10 mm/s
   The maximum force measured during the stage lowering step is a measure of the adhesive force of the viscoelastic particle and the hair fiber. The higher the force the greater the adhesive properties between the hair fiber and the viscoelastic particle. As shown in FIG. 2.

4. Dried Storage (G′) and Dried Loss (G″) Modulus
Equipment: Haake RS-600 Rheostress with 35 mm/4° cone and plate (or equivalent)
All measurements done with plate temperature of 25° C.
   a) The viscoelastic particle is placed in dish with wall height of 7 mm or less and top levelled to wall height with spatula.
   b) The viscoelastic particle is set at ambient room conditions (20-25° C.) to dry for 5 days (112-128 hours) to remove all or part of the volatile swelling solvent (whatever is removed under these conditions).
   c) Two oscillatory stress ramps of the dried viscoelastic particle are run, one at 0.1 rad/s and one at 100 rad/s from 0 Pa to 50 Pa.
   d) G′ (Pa·s) and G″ (Pa·s) are recorded in the viscoelastic region for both test runs.
   e) The viscoelastic windows are constructed by plotting the four coordinates: (1) G′ at 0.1 rad/s, G″ at 0.1 rad/s, (2) G′ at 100 rad/s, G″ at 0.1 rad/s, (3) G′ at 0.1 rad/s, G″ at 100 rad/s, (4) G′ at 100 rad/s, G″ at 100 rad/s on a log-log cross plot of G′ and G″.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

| | All ingredients in % as added | | | | | | |
|---|---|---|---|---|---|---|---|
| | Leave on Treatment Composition Examples | | | | | | |
| | Leave on | Leave on | Leave on | Leave on | Leave on | Leave on | Leave on |
| | Treatment | Treatment | Treatment | Treatment | Treatment | Treatment | Treatment |
| Ingredient | 1 | 2 | 3 | 4 | 5 | 6 | 7 |
| Water | QS | QS | QS | QS | QS | QS | QS |
| SD Alcohol 40 ¹ | | 50.0000 | | | | | |
| Polyacrylamide & | | | 1.0000 | | | | |
| C13-14 Isoparaffin & | | | | | | | |
| Laureth-7 ² | | | | | | | |
| Glyoxylic Acid ³ | | | | | 7.5000 | | |
| Dehydroxanthan Gum ⁴ | | 0.7500 | | | | | |
| HydroxyethylCellulose ⁵ | | | | 0.3112 | | 0.3112 | |
| PEG-2M⁶ | | | | 0.2075 | | 0.2075 | |
| Quaternium-18 ⁷ | | | | 0.3112 | | 0.3112 | |
| Sodium Polyacrylate | 0.5800 | | | | | | 0.7000 |
| Starch ⁸ | | | | | | | |
| Stearamidopropyl | | | | 0.4149 | | 0.4149 | |
| Dimethylamine ⁹ | | | | | | | |
| Dimethicone/Vinyl | 36.3600 | 18.1810 | 27.0000 | 36.3600 | 20.0000 | 36.3600 | 18.1810 |
| Dimethicone | | | | | | | |
| Crosspolymer ¹⁰ | | | | | | | |
| Bis-PEG/PPG-20/20 | | 0.3000 | | | | | |
| Dimethicone ¹⁴ | | | | | | | |
| Cetearyl Alcohol & | | | | 0.2075 | | 0.2075 | |
| Polysorbate 60 ¹⁵ | | | | | | | |
| 10,000 cSt | | | 1.5000 | | | | |
| Dimethicone ¹⁶ | | | | | | | |
| Cetyl Alcohol ¹⁷ | | | | 0.3984 | | 0.3984 | |
| Glycerine ¹⁸ | 0.3000 | | | | | | |
| Caffeine ¹⁹ | | 0.9375 | | | | | |
| Niacinamide ²⁰ | | 3.1250 | | | | | |
| D-Panthenol ²¹ | | 0.1875 | | | | | |
| Stearyl Alcohol ²² | | | | 0.2654 | | 0.2654 | |
| PEG-10 Dimethicone²³ | | | | | 0.5000 | | 1.0100 |
| Cyclopentasiloxane²⁴ | | | | | 5.0000 | | |
| PEG-40 Hydrogenated | | | | | | | 0.3250 |
| Castor Oil²⁵ | | | | | | | |
| Straightening Actives | | | | | | 5.0000 | |
| Fragrance | 0.7000 | 0.3000 | 1.0000 | 0.7000 | 0.5000 | 0.7000 | 0.1300 |
| Preservatives, pH | Up to 2% | Up to 2% | Up to 2% | Up to 2% | Up to 2% | Up to 2% | Up to 2% |
| adjusters | | | | | | | |

¹ SD Alcohol 40B (200 Proof) supplier Equistar Chemicals
² Sepigel 305, 45% active, Supplier Seppic Inc.
³ Glyoxo High pure 50, 50% active, Supplier Clariant
⁴ Amaze XT, 100% active, Supplier Akzo Nobel
⁵ Natrosol 250 HR/HHR, 100% active, Supplied byAshland Specialty Ingredients
⁶Polyox WSR N-10, 100% active, Supplied by Dow Consumer & Industrial Solutions
⁷ Varisoft 442 100p, 100% active, Supplied by Goldschmidt Chemical.
⁸ Makimousse 12, 100% active, Supplier KOBO Products
⁹ Incromine SD-PA-(MH), 100% active, Supplier Croda
¹⁰ SFE839, 5.5% active, 94.5% cyclopentasiloxane Supplier Momentive Inc.
¹⁴ Abil B 8832, 100% Active, Supplier Evonik Industries AG
¹⁵ Lipowax P, 100% Active, Supplier Lipo Chemicals
¹⁶ TSF451 1MA, 100% Active, Supplier Dow Corning Toray Co Ltd
¹⁷ Cetyl Alcohol, 100% active, Supplier Sigma Aldrich
¹⁸ Glycerine, 100% active, Supplier Spectrum Chemicals
¹⁹ Caffeine USP, 100% active, Supplier BASF Pharmachemikalien Gmbh Kg
²⁰ Niacinamide USP FCC, 100% active. Supplier DSM Nutritional Products Inc
²¹ D-Panthenol, 100% active, Supplier DSM Nutritional Products Inc
²² Stearyl Alcohol, 100% active, Supplier Sigma Aldrich
²³ Dow Corning ES-5612 Formulation Aid, 100% Active, Supplier Dow Corning
²⁴SF1202, 100% Active, Supplier Momentive
²⁵Cremophor CO 40, 100% Active, Supplier BASF

| | | EVALUATION RESULTS | |
|---|---|---|---|
| | COMPARATIVE EXAMPLES | Hair Weigh- | Visual Fiber |
| | LEAVE-ON HAIR COMPOSITION | down | Alignment |
| A | Formula 1 (Control) | No | Fibers are |
| | | | aligned |
| B | Formula 1 substituting SFE839 | | Fibers are |
| | with DC9040 | | not aligned |
| C | Formula 1 substituting SFE839 | | Fibers are |
| | with KSG-320 | | not aligned |
| D | Formula 1 substituting SFE839 | Yes | |
| | with KSG-330 | | |

Those skilled in the art can combine the ingredients in the examples using standard laboratory mixing and heating equipment to form homogenous products.

TABLE 2
1-10 rating scale

| | Experimental (containing viscoelastic particles) |
|---|---|
| Atribute | vs Commercial Leave On Hair Treatment containing |
| n = 34 panelist | silicone fluid and no viscoelastic particles |
| Less Greasy Look | Experimental Preferred (3.4 vs 5.2 (lower is better)) |
| | p = <0.0001 |
| Less Greasy Feel | Experimental Preferred (3.1 vs 4.8 (lower is better)) |
| | p = 0.0002 |
| Increase in Volume | Experimental Preferred (5.9 vs 4.9 (higher is better)) |
| | p = 0.1212 |
| Less Weigh Down | Experimental Preferred (4.6 vs 6.8 (lower is better)) |
| | p = <0.0001 |

TABLE 3
Visual Grading of switches

| | | | |
|---|---|---|---|
| Sample | 0.4 g/g hair | 1.0 g/g hair | 2.0 g/g hair |

Alignment Rating

| | | | |
|---|---|---|---|
| Viscoelastic particle | 3 | 3 | 1 |
| (Formula 1) (2%) | | | |
| Silicone Fluid | 2 | 2 | 1 |
| (Formula 2)(2%) | | | |

Greasiness Rating

| | | | |
|---|---|---|---|
| Viscoelastic particle | 1 | 1 | 3 |
| (Formula 1) (2%) | | | |
| Silicone Fluid | 2 | 3 | 4 |
| (Formula 2)(2%) | | | |

Frizz Rating

| | | | |
|---|---|---|---|
| Viscoelastic particle | 2 | 2 | 1 |
| (Formula 1) (2%) | | | |
| Silicone Fluid | 3 | 1 | 1 |
| (Formula 2)(2%) | | | |

| | | |
|---|---|---|
| Alignment | Greasiness | Frizz |
| 1 = Excellent | 1 = None | 1 = No Frizz |
| 2 = Very Good | 2 = Slight | 2 = Slight |
| 3 = Good | 3 = Minimal | 3 = Minimal |
| 4 = Fair | 4 = Moderate | 4 = Moderate |
| 5 = Poor | 5 = Heavy | 5 = Lots of Frizz |

### Compositions Used in Visual Grading Evaluations:

| | Wt % Formula 1 | Wt % Formula 2 |
|---|---|---|
| Water | 62.76 | 97.12 |
| Makimousse 12 | 0.58 | 0.58 |
| Glycerin | 0.30 | 0.30 |
| Viscoelastic particle SFE839 | 36.36 | — |
| Silicone Fluid (Momentive | — | 2.00 |
| Y14945 amino fluid) | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A hair care composition comprising:
a. from about 1 wt % to about 80 wt % of viscoelastic particles, wherein the viscoelastic particles comprise from about 0.5 wt % to about 50 wt % crosslinked polymer swelled in about 50 wt % to about 99.5 wt % volatile solvent;
b. from about 99 wt % to about 20 wt % of a carrier;
wherein the viscoelastic particle has a dried storage modulus of about 85 to about 10,000 Pascals, and a dried loss modulus of about 50 to about10,000 Pascals; and wherein the carrier comprises less than about 2 wt % non-volatile solvent.

2. The hair care composition of claim 1, further comprising up to about 5 wt % of a surfactant.

3. The hair care composition of claim 1, further comprising a thickener.

4. The hair care composition of claim 1, wherein the viscoelastic particles are silicone elastomers.

5. The hair care composition of claim 1, comprising from about 5 wt % to about 50 wt % of viscoelastic particles.

6. The hair care composition of claim 1, wherein the viscoelastic particles comprise from about 5 wt % to about 40 wt % crosslinked polymer and from about 95 wt % to about 60 wt % solvent.

7. The hair care composition of claim 1, wherein the viscoelastic particles comprises volatile solvents selected from the group consisting of volatile siloxanes, volatile hydrocarbons, and mixtures thereof.

8. The hair care composition of claim 1, wherein the carrier is selected from the group consisting of water, silicones, organic solvents, hydrocarbons, and mixtures thereof.

9. The hair care composition of claim 1, wherein the dried elastic modulus is from about 3,000 to about 5,000 and the dried Loss modulus is from about 500- to about 1000.

10. The hair care composition of claim 1, further comprising up to about 2 wt % of a styling polymer.

11. The hair care composition of claim 1, further comprising a material selected from the group consisting of anti-dandruff actives, antimicrobial materials, skin and scalp actives, vitamins, hair growth actives, conditioning materials, fragrances, pigments, colorings/colorants, essential oils, sensates, antifoaming agents, antioxidants, binders, biological additives, buffering agents, bulking agents, chelating agents, chemical additives, film formers or materials, opacifying agents, pH adjusters, propellants, reducing agents, and surfactants.

12. The hair care composition of claim 11, wherein the composition comprises from about 0.1 wt % to about 2 wt % of a film forming polymer.

13. The hair care composition of claim 12, wherein the film forming polymer is selected from the group consisting of acrylates, acylamides, silicones, polyvinyl compounds, polyquaterium compounds, elastomeric materials, latexes, polyurethanes, polyethylenes, polystyrenes, nylon compound, and wax based compounds.

14. The hair care composition of claim 12, wherein the film forming polymer is a naturally derived film forming polymer selected from the group consisting of shellac, rubber, cellulose, chitosans, polysaccarides, and proteins.

15. The hair care composition of claim 1, wherein the composition further comprises an associative thickener.

16. A method of reducing hair frizz by applying the composition of claim 1 to the hair.

17. The method of claim 16, further comprising the step of heat styling the hair.

18. The method of claim 17, wherein the heat styling includes flat ironing the hair.

19. A method of improving hair shine by applying the composition of claim 1 to the hair.

20. The hair care composition of claim 1, wherein the carrier is aqueous.
